(19) United States
(12) Patent Application Publication
(10) Pub. No.: US 2004/0253689 A1
(43) Pub. Date: Dec. 16, 2004

(54) Mutants of human insulin-like growth factor binding protein-3 (igfbp-3) and uses thereof

(76) Inventor: Mathew Rechler, Washington, DC (US)

Correspondence Address: LEYDIG, VOIT & MAYER, LTD., TWO PRUDENTIAL PLAZA, SUITE 4900, CHICAGO, IL, 60601, US

(21) Appl. No.: 10/499,379

(22) PCT Filed: Dec. 17, 2002
(86) PCT No.: PCT/US02/40561

Related U.S. Application Data
(60) Provisional application No. 60/341,920, filed on Dec. 17, 2001.

(51) Int. Cl. C12P 19/34

(52) U.S. Cl. 435/91.2

(57) ABSTRACT

An isolated or purified nucleic acid molecule consisting essentially of a nucleotide sequence encoding a mutant human IGFBP-3, which can inhibit DNA synthesis, can induce apoptosis, binds to neither human insulin growth factor-I (IGF-I), nor human insulin growth factor-II (IGF-II), and comprises a mutation at Y57; a vector comprising the same, a cell comprising and expressing the same, optionally in the form of a vector; an isolated or purified polypeptide molecule consisting essentially of an amino acid sequence encoding a mutant human IGFBP-3, which can inhibit DNA synthesis, can induce apoptosis, binds to neither human IGF-I nor human IGF-II and comprises a mutation at Y57; a composition comprising the same; and a method of inducing apoptosis in a cell, which method comprises administering to the cell the nucleic acid molecule or polypeptide molecule, in an amount sufficient to induce apoptosis in the cell, whereupon apoptosis is induced in the cell.

### FIELD OF THE INVENTION

This invention pertains to mutants of human IGFBP-3 and uses thereof.

### BACKGROUND OF THE INVENTION

The American Cancer Society estimates the lifetime risk that an individual will develop cancer is 1 in 2 for men and 1 in 3 for women. Prostate cancer is the most common non-cutaneous malignancy diagnosed in men in the United States, accounting for over 40,000 deaths annually (Parker et al, J. Clin. Cancer. 46:5, 1996). The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., deletions, substitutions, etc.) can be inherited from generation to generation, thereby imparting an individual with a genetic predisposition to develop cancer.

Therefore, there remains a need for a new, safe and effective method of treating cancer. The present invention provides such a method, as well as isolated or purified nucleic acid molecules, optionally in the form of vectors, isolated or purified polypeptide molecules, and related compositions, which optionally comprise other anti-cancer agents, for use in the method.

### BRIEF SUMMARY OF THE INVENTION

The present invention provides an isolated or purified nucleic acid molecule consisting essentially of a nucleotide sequence encoding a mutant human IGFBP-3, which can inhibit DNA synthesis, can induce apoptosis, binds to neither human insulin growth factor-I (IGF-I) nor human insulin growth factor-II (IGF-II), and comprises a mutation at Y57. A vector comprising such an isolated or purified nucleic acid molecule is also provided as is a cell comprising and expressing the isolated or purified nucleic acid molecule, optionally in the form of a vector.

Further provided is an isolated or purified polypeptide molecule consisting essentially of an amino acid sequence encoding a mutant human IGFBP-3, which can inhibit DNA synthesis, can induce apoptosis, binds to neither human IGF-I nor human IGF-II, and comprises a mutation at Y57. A composition comprising an isolated or purified polypeptide molecule consisting essentially of an amino acid sequence encoding a mutant human IGFBP-3, which can inhibit DNA synthesis, can induce apoptosis, binds to neither IGF-I nor IGF-II, is also provided.

Still further provided is a method of inducing apoptosis in a cell. The method comprises administering to the cell:

(a) an isolated or purified nucleic acid molecule consisting essentially of a nucleotide sequence encoding a mutant human IGFBP-3, which can inhibit DNA synthesis, can induce apoptosis, and binds to neither human IGF-I nor human IGF-II, optionally in the form of a vector, or

(b) an isolated or purified polypeptide molecule consisting essentially of an amino acid sequence encoding a mutant human IGFBP-3, which can inhibit DNA synthesis, can induce apoptosis, and binds to neither human IGF-I nor human IGF-II, in an amount sufficient to induce apoptosis in the cell, whereupon apoptosis is induced in the cell.

### BRIEF DESCRIPTION OF THE DRAWINGS

FIG. 1 (top) provides the amino acid sequences of the IGF-binding domain of human insulin-like growth factor binding protein-5 (hIGFBP-5; residues 43-76; SEQ ID NO: 1), human IGFBP-3 (hIGFBP-3; residues 50-83; SEQ ID NO: 2), and the mutants 6m-hIGFBP-3, 4m-hIGFBP-3 and 2m-hIGFBP-3, the mutated residues of which and the corresponding residues in hIGFBP-5 and hIGFBP-3 are boxed.

FIG. 1 (bottom) provides the schematic diagram of plasmid pRSV-Sec-BP3, which was used to transfect stably Chinese hamster ovary (CHO)-K1 cells and express wild-type hIGFBP-3.

### DETAILED DESCRIPTION OF THE INVENTION

The present invention provides an isolated or purified nucleic acid molecule consisting essentially of a nucleotide sequence encoding a mutant human IGFBP-3, which can inhibit DNA synthesis, can induce apoptosis, binds to neither human IGF-I, nor human IGF-II, and comprises a mutation at Y57. By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” “Nucleic acid molecule” is intended to encompass a polymer of DNA or RNA, i.e., a polynucleotide, which can be single-stranded or double-stranded and which can contain non-natural or altered nucleotides. Desirably, the isolated or purified nucleic acid molecule does not contain any introns or portions thereof.

Desirably, the isolated or purified nucleic acid molecule additionally comprises a mutation of at least one of the amino acids selected from the group consisting of I56, R75, L77, L80 and L81. Preferably, the mutation is a substitution of at least one of the amino acids selected from the group consisting of I56, Y57, R75, L77, L80 and L81 with another amino acid that compromises the ability of IGFBP-3 to bind to IGF-I and IGF-II. Preferably, the amino acid that compromises the ability of IGFBP-3 to bind to IGF-I and IGF-II is alanine. In a preferred embodiment, all of I56, Y57, R75, L77, L80 and L81 are substituted with alanine.

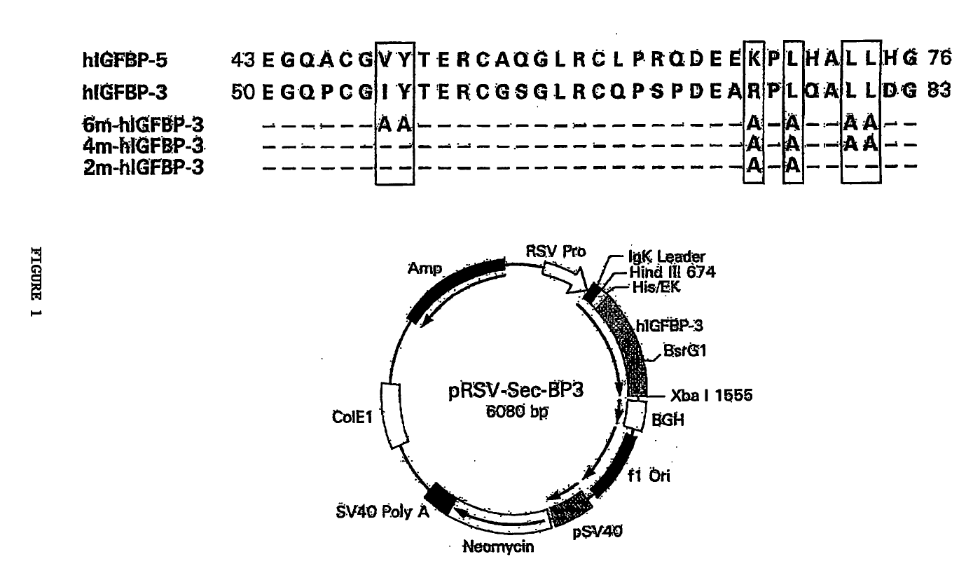

The entire sequence of the human IGFBP-3 clone is known (Genbank Accession No. M31159; see, also, Wood et al., Mol. Endocrinol. 2(12): 1176-1185 (1988)). See also the top of FIG. 1, which provides the amino acid sequences of the IGF-binding domain of human IGFBP-5 (residues 43-76; SEQ ID NO: 1), human IGFBP-3 (residues 50-83; SEQ ID NO: 2), and the mutants 6m-hIGFBP-3, 4m-hIGFBP-3 and 2m-hIGFBP-3, the mutated residues of which and the corresponding residues in hIGFBP-5 and hIGFBP-3 are boxed. With respect to the above, one of ordinary skill in the art knows how to generate mutations, e.g., insertions, deletions, substitutions and/or inversions, in a given nucleic acid molecule. See, for example, the references cited herein under “Example.”

While the above-described mutated nucleic acid molecules can be generated in vivo and then isolated or purified, alternatively they can be synthesized. Methods of nucleic acid synthesis are known in the art. See, e.g., the references cited herein under “Example.”

In view of the above, the present invention also provides a vector comprising an above-described isolated or purified nucleic acid molecule, optionally as part of an encoded fusion protein. A nucleic acid molecule as described above can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987) and the references cited herein under “Example”). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

Constructs of vectors, which are circular or linear, can be prepared to contain an entire nucleic acid sequence as described above or a portion thereof ligated to a replication system functional in a prokaryotic or eukaryotic host cell. Replication systems can be derived from ColE1, 2 mμ plasmid, λ, SV40, bovine papilloma virus, and the like.

In addition to the replication system and the inserted nucleic acid, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. A preferred cloning vector is selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM 1149, also can be used. Examples of plant expression vectors include pBI101, pBI101.2, pBI101.3, pBI121 and pBIN19 (Clontech). Examples of animal expression vectors include pEUK-C1, pMAM and pMAMneo (Clontech).

An expression vector can comprise a native or nonnative promoter operably linked to an isolated or purified nucleic acid molecule as described above. The selection of promoters, e.g., strong, weak, inducible, tissue-specific and developmental-specific, is within the skill in the art. Similarly, the combining of a nucleic acid molecule as described above with a promoter is also within the skill in the art.

Optionally, the isolated or purified nucleic acid molecule, upon linkage with another nucleic acid molecule, can encode a fusion protein. The generation of fusion proteins is within the ordinary skill in the art (see, e.g., references cited under “Example”) and can involve the use of restriction enzyme or recombinational cloning techniques (see, e.g., Gateway™ (Invitrogen, Carlsbad, Calif.)). See, also, U.S. Pat. No. 5,314,995.

Also in view of the above, the present invention provides a cell comprising and expressing an isolated or purified nucleic acid molecule, optionally in the form of a vector, as described above. Examples of cells include, but are not limited to, a human cell, a human cell line, E. coli (e.g., E. coli TB-1, TG-2, DH5α, XL-Blue MRF′ (Stratagene), SA2821 and Y1090), B. subtilis, P. aerugenosa, S. cerevisiae, N. crassa, insect cells (e.g., Sf9, Ea4) and others set forth herein below.

An isolated or purified polypeptide molecule consisting essentially of an amino acid sequence encoding a mutant human IGFBP-3, which can inhibit DNA synthesis, can induce apoptosis, binds to neither human IGF-I nor human IGF-II, and comprises a mutation at Y57, is also provided. By “isolated” is meant the removal of a polypeptide from its natural environment. By “purified” is meant that a given polypeptide has been increased in purity, where “purity” is a relative term, not “absolute purity.” Desirably, the isolated or purified polypeptide molecule additionally comprises a mutation of at least one of the amino acids selected from the group consisting of I56, R75, L77, L80, and L81. Preferably, the mutant human IGFBP-3 comprises a substitution of at least one of the amino acids selected from the group consisting of I56, Y57, R75, L77, L80 and L81 with another amino acid that compromises the ability of IGFBP-3 to bind to IGF-I and IGF-II. Preferably, the amino acid that compromises the ability of the IGF-binding domain of IGFBP-3 to bind to IGF-I and IGF-II is alanine. In a preferred embodiment, all of I56, Y57, R75, L77, L80 and L81 are substituted with alanine.

The isolated or purified polypeptide molecule can be optionally glycosylated, amidated, carboxylated, phosphorylated, esterified, N-acylated or converted into an acid addition salt. Methods of protein modification (e.g., glycosylation, amidation, carboxylation, phosphorylation, esterification, N-acylation, and conversion into acid addition salts) are known in the art.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo, given that the D-amino acids are not recognized by naturally occurring proteases.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2^{nd} ed. (Cold Spring Harbor Laboratory, 1982); Sambrook et al., Molecular Cloning A Laboratory Manual, 2^{nd} ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, and BHK cell lines, and the like. The ordinarily skilled artisan is, of course, aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

Alternately, the mutant polypeptide can be synthesized using standard peptide synthesizing techniques well-known to those of ordinary skill in the art (e.g., as summarized in Bodanszky, Principles of Peptide Synthesis (Springer-Verlag, Heidelberg: 1984)). In particular, the polypeptide can be synthesized using the procedure of solid-phase synthesis (see, e.g., Merrifield, J. Am. Chem. Soc. 85: 2149-54 (1963); Barany et al., Int. J. Peptide Protein Res. 30: 705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

If desired, the mutant polypeptides of the invention can be modified, for instance, by glycosylation, amidation, carboxylation, or phosphorylation, or by the creation of acid addition salts, amides, esters, in particular C-terminal esters, and N-acyl derivatives of the polypeptides of the invention. The polypeptides also can be modified to create polypeptide derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus.

Thus, a fusion protein and a conjugate comprising an above-described isolated or purified polypeptide molecule or fragment thereof and a therapeutically or prophylactically active agent can be generated. “Prophylactically” as used herein does not necessarily mean prevention, although prevention is encompassed by the term. Prophylactic activity also can include lesser effects, such as inhibition of the onset of cancer. Preferably, the active agent is an anti-cancer agent. Methods of conjugation are known in the art. In addition, conjugate kits are commercially available. For examples of methods of conjugation and conjugates see, e.g., Hermanson, G. T., Bioconjugate Techniques, 1996, Academic Press, San Diego, Calif.; U.S. Pat. Nos. 6,013,779; 6,274,552 and 6,080,725; and Ragupathi et al., Glycoconjugate Journal 15: 217-221 (1998).

The present invention also provides a composition comprising an above-described isolated or purified polypeptide molecule (or conjugate or fusion protein thereof). The composition can be a pharmaceutical composition, additionally comprising a carrier and, optionally, an anti-cancer agent. Pharmaceutical compositions containing the present inventive polypeptide molecule (or conjugate or fusion protein thereof) can comprise more than one active ingredient, such as more than one polypeptide molecule (or conjugate or fusion protein thereof). The pharmaceutical composition can alternatively comprise a polypeptide molecule (or conjugate or fusion protein thereof) in combination with other pharmaceutically active agents or drugs.

The anti-cancer agent can be a chemotherapeutic agent, e.g., a polyamine or an analogue thereof. Examples of therapeutic polyamines include those set forth in U.S. Pat. Nos. 5,880,161, 5,541,230 and 5,962,533, Saab et al., J. Med. Chem. 36: 2998-3004 (1993), Bergeron et al., J. Med. Chem. 37(21): 3464-3476 (1994), Casero et al., Cancer Chemother. Pharmacol 36: 69-74 (1995), Bernacki et al., Clin. Cancer Res. 1: 847-857 (1995); Bergeron et al., J. Med. Chem. 40: 1475-1494 (1997); Gabrielson et al., Clinical Cancer Res. 5: 1638-1641 (1999), and Bergeron et al., J. Med. Chem. 43: 224-235 (2000), which can be administered alone or in combination with other active agents, such as anti-cancer agents, e.g., cis-diaminedichloroplatinum (II) and 1,3-bis(2-chloroethyl)-1-nitrosourea. Other anti-cancer agents include, for example, TGF-β, anti-estrogens, retinoids, 1,25-dihydroxyvitamin D3, ceramide, and antimycin A. Irradiation and surgical procedures as are known in the art also can be employed in combination with the present inventive method.

The carrier can be any suitable carrier. Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. It will be appreciated by one of skill in the art that, in addition to the following described pharmaceutical compositions, the present inventive polypeptide molecule (or conjugate or fusion protein thereof) can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide molecule (or conjugate or fusion protein thereof), as well as by the particular method used to administer the polypeptide molecule (or conjugate or fusion protein thereof). Accordingly, there are a variety of suitable formulations of the pharmaceutical composition of the present invention. The following formulations for oral, aerosol, parenteral, subcutaneous, intravenous, intramuscular, interperitoneal, rectal, and vaginal administration are exemplary and are in no way limiting. One skilled in the art will appreciate that these routes of administering the polypeptide molecule (or conjugate or fusion protein thereof) of the present invention are known, and, although more than one route can be used to administer a particular polypeptide molecule (or conjugate or fusion protein thereof), a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Topical formulations are well-known to those of skill in the art. Such formulations are particularly suitable in the context of the present invention for application to the skin.

Formulations suitable for oral administration can consist of (a) liquid solutions, such as an effective amount of the polypeptide molecule (or conjugate or fusion protein thereof) dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The polypeptide molecule (or conjugate or fusion protein thereof), alone or in combination with each other and/or with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The polypeptide molecule (or conjugate or fusion protein thereof) can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the polypeptide molecule (or conjugate or fusion protein thereof), or compositions comprising such polypeptide molecule (or conjugate or fusion protein thereof), can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

One of ordinary skill in the art will readily appreciate that the polypeptide molecule (or conjugate or fusion protein thereof) of the present invention can be modified in any number of ways, such that the therapeutic efficacy of the polypeptide molecule (or conjugate or fusion protein thereof) is increased through the modification. For instance, the polypeptide molecule could be conjugated either directly or indirectly through a linker to a targeting moiety. The practice of conjugating polypeptide molecules to targeting moieties is known in the art. See, for instance, Wadwa et al., J. Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the polypeptide molecule (or conjugate or fusion protein thereof) to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used herein, refers to any agent or molecule that bridges the polypeptide molecule (or conjugate or fusion protein thereof) to the targeting moiety. One of ordinary skill in the art recognizes that sites on the polypeptide molecule (or conjugate or fusion protein thereof), which are not necessary for the function of the compound or inhibitor, are ideal sites for attaching a linker and/or a targeting moiety, provided that the linker and/or targeting moiety, once attached to the polypeptide molecule (or conjugate or fusion protein thereof), do(es) not interfere with the function of the polypeptide molecule (or conjugate or fusion protein thereof).

Alternatively, the polypeptide molecule (or conjugate or fusion protein thereof) of the present invention can be modified into a depot form, such that the manner in which the polypeptide molecule (or conjugate or fusion protein thereof) is released into the body to which it is administered is controlled with respect to time and location within the body (see, for example, U.S. Pat. No. 4,450,150). Depot forms of the polypeptide molecule (or conjugate or fusion protein thereof) can be, for example, an implantable composition comprising the polypeptide molecule (or conjugate or fusion protein thereof) and a porous material, such as a polymer, wherein the polypeptide molecule (or conjugate or fusion protein thereof) is encapsulated by or diffused throughout the porous material. The depot is then implanted into the desired location within the body and the polypeptide molecule (or conjugate or fusion protein thereof) is released from the implant at a predetermined rate by diffusing through the porous material.

The present invention also provides a method of inducing apoptosis in a cell. The method comprises administering to the cell:

(a) an isolated or purified nucleic acid molecule consisting essentially of a nucleotide sequence encoding a mutant human IGFBP-3, which can inhibit DNA synthesis, can induce apoptosis, and binds to neither human IGF-I nor human IGF-II, optionally in the form of a vector, or

(b) an isolated or purified polypeptide molecule consisting essentially of an amino acid sequence encoding a mutant human IGFBP-3, which can inhibit DNA synthesis, can induce apoptosis, and binds to neither human IGF-I nor human IGF-II, in an amount sufficient to induce apoptosis in the cell, whereupon apoptosis in induced in the cell.

In a preferred embodiment, the cell of the present inventive method is in a host. The benefits of the invention, that is, induction of apoptosis, that can be observed and realized at the cellular level are also observable and realized in the host. The host can be any host, including for example, bacteria, yeast, fungi, plants, and mammals. Preferably, the host is a mammal. For purposes of the present invention, mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In one embodiment of the present invention, the host is afflicted with a cancer. The cancer can be a cancer selected from the group consisting of prostate cancer, colorectal cancer, lung cancer, and childhood-onset leukemia Treatment of the host in accordance with the present inventive method of inducing apoptosis will result in treatment of cancer in the host.

Preferred routes of administration in the method of inducing apoptosis include oral, aerosol, parenteral, subcutaneous, intravenous, intramuscular, interperitoneal, rectal, and vaginal administration, and these routes have been discussed herein. Also preferred is that the polypeptide molecule (or conjugate or fusion protein thereof) or the nucleic acid molecule of the present invention is administered to the cell in vitro. As used herein, the term “in vitro” means that the cell is not in a living organism. In this case, it is desirable that the cell to which the mutant IGFBP-3 polypeptide or nucleic acid molecule was administered is subsequently administered to the host. It is also preferred that the polypeptide molecule (or conjugate or fusion protein thereof) or nucleic acid molecule of the present invention is administered to the cell in vivo. As used herein, the term “in vivo” means that the cell is a part of a living organism or is the living organism.

In the instance that the cell is in a host afflicted with cancer, it is preferred that the polynucleotide or nucleic acid molecule of the mutant IGFBP-3 is administered intratumorally or peritumorally. A preferred manner of administering a polypeptide molecule or nucleic acid molecule of the present invention is by targeting to a cancer cell. In this regard, examples of cancer-specific, cell-surface molecules include placental alkaline phosphatase (testicular and ovarian cancer), pan carcinoma (small cell lung cancer), polymorphic epithelial mucin (ovarian cancer), prostate-specific membrane antigen, α-fetoprotein, B-lymphocyte surface antigen (B-cell lymphoma), truncated EGFR (gliomas), idiotypes (B-cell lymphoma), gp95/gp97 (melanoma), N-CAM (small cell lung carcinoma), cluster w4 (small cell lung carcinoma), cluster 5A (small cell carcinoma), cluster 6 (small cell lung carcinoma), PLAP (seminomas, ovarian cancer, and non-small cell lung cancer), CA-125 (lung and ovarian cancers), ESA (carcinoma), CD19, 22 or 37 (B-cell lymphoma), 250 kD proteoglycan (melanoma), P55 (breast cancer), TCR-IgH fusion (childhood T-cell leukemia), blood group A antigen in B or O type individual (gastric and colon tumors), and the like. See, e.g., U.S. Pat. No.6,080,725 for other examples.

Examples of cancer-specific, cell-surface receptors include erbB-2, erbB-3, erbB-4, IL-2 (lymphoma and leukemia), IL-4 (lymphoma and leukemia), IL-6 (lymphoma and leukemia), MSH (melanoma), transferrin (gliomas), tumor vasculature integrins, and the like. Preferred cancer-specific, cell-surface receptors include erbB-2 and tumor vasculature integrins, such as CD11a, CD11b, CD11c, CD18, CD29, CD51, CD61, CD66d, CD66e, CD106, and CDw145.

There are a number of antibodies to cancer-specific, cell-surface molecules and receptors that are known. C46 Ab (Amersham) and 85A12 Ab (Unipath) to carcino-embryonic antigen, H17E2 Ab (ICRF) to placental alkaline phosphatase, NR-LU-10 Ab (NeoRx Corp.) to pan carcinoma, HMFCl Ab (ICRF) to polymorphic epithelial mucin, W14 Ab to B-human chorionic gonadotropin, RFB4 Ab (Royal Free Hospital) to B-lymphocyte surface antigen, A33 Ab (Genex) to human colon carcinoma, TA-99 Ab (Genex) to human melanoma, antibodies to c-erbB2 (JP 7309780, JP 8176200 and JP 7059588), and the like. ScAbs can be developed, based on such antibodies, using techniques known in the art (see for example, Bind et al., Science 242: 423-426 (1988), and Whitlow et al., Methods 2(2): 97-105 (1991)).

Generally, when a mutant human IGFBP-3 (or a conjugate or fusion protein thereof) is administered to an animal, such as a mammal, in particular a human, it is desirable that the mutant IGFBP-3 be administered in a dose of from about 1 to about 100 or higher μg/kg body weight/treatment when given parenterally. Higher or lower doses may be chosen in appropriate circumstances. For instance, the actual dose and schedule can vary depending on whether the composition is administered in combination with other pharmaceutical compositions, for example, or depending on interindividual differences in pharmacokinetics, drug disposition, and metabolism. One skilled in the art easily can make any necessary adjustments in accordance with the necessities of the particular situation.

Those of ordinary skill in the art can easily make a determination of the amount of an above-described isolated and purified nucleic acid molecule to be administered to an animal, such as a mammal, in particular a human. The dosage will depend upon the particular method of administration, including any vector or promoter utilized. For purposes of considering the dose in terms of particle units (pu), also referred to as viral particles, it can be assumed that there are 100 particles/pfu (e.g., 1×10¹² pfu is equivalent to 1×10¹⁴ pu). An amount of recombinant virus, recombinant DNA vector or RNA genome sufficient to achieve a tissue concentration of about 10² to about 10¹² particles per ml is preferred, especially of about 10⁶ to about 10¹⁰ particles per ml. In certain applications, multiple daily doses are preferred. Moreover, the number of doses will vary depending on the means of delivery and the particular recombinant virus, recombinant DNA vector or RNA genome administered.

The human IGFBP-3 mutants of the present invention also can be used to develop therapeutic agents that can selectively activate the same antiproliferative pathway in tumor cells. Such therapeutic agents can then be used in the treatment of cancer.

### EXAMPLES

The following examples further illustrate the invention but, of course, should not be construed as in any way limiting its scope.

The following references, to the extent that they provide exemplary procedural or other details supplementary to those set forth herein, are specifically incorporated herein by reference:

Birren et al., Genome Analysis: A Laboratory Manual Series, Volume 1, Analyzing DNA, Cold Spring Harbor Laboratory Press, Cold Spring Harbor, N.Y. (1997),

Birren et al., Genome Analysis: A Laboratory Manual Series, Volume 2, Detecting Genes, Cold Spring Harbor Laboratory Press, Cold Spring Harbor, N.Y. (1998),

Birren et al., Genome Analysis: A Laboratory Manual Series, Volume 3, Cloning Systems, Cold Spring Harbor Laboratory Press, Cold Spring Harbor, N.Y. (1999),

Birren et al., Genome Analysis: A Laboratory Manual Series, Volume 4, Mapping Genomes, Cold Spring Harbor Laboratory Press, Cold Spring Harbor, N.Y. (1999),

Harlow et al., Antibodies: A Laboratory Manual, Cold Spring Harbor Laboratory Press, Cold Spring Harbor, N.Y. (1988),

Harlow et al., Using Antibodies: A Laboratory Manual, Cold Spring Harbor Laboratory Press, Cold Spring Harbor, N.Y. (1999),

Hoffman, Cancer and the Search for Selective Biochemical Inhibitors, CRC Press (1999),

Pratt, The Anticancer Drugs, 2nd edition, Oxford University Press, NY (1994), and

Sambrook et al., Molecular Cloning: A Laboratory Manual, 2nd edition, Cold Spring Harbor Laboratory Press, Cold Spring Harbor, N.Y. (1989).

Materials—Plasmids pRc/RSV and pcDNA3.1/His A, anti-Xpress antibody, and ProBond Resin were purchased from Invitrogen (Carlsbad, Calif.). A human IGFBP-3 cDNA clone (Genbank Accesion No. M31159) was provided by William Wood (Genentech, South San Francisco, Calif.) (Wood et al. (1988), supra). The QuikChange Site-Directed Mutagenesis kit was obtained from Stratagene (LaJolla, Calif.).

Recombinant hIGFBP-3 synthesized in NSO mouse myeloma cells was obtained from R&D systems (Minneapolis, Minn.) and used as reference standard. Leu⁶⁰-IGF-I expressed in Eschericia coli was kindly provided by Celtrix Pharmaceuticals, Inc. (San Jose, Calif.) (Wu et al., J. Cell Biochem. 77(2), 288-97 (2000)). Monoclonal antibodies to the N-terminus (antibody #3, amino acids 1-97) or C-terminus (antibody #1, residues 98-264) of hIGFBP-3 (Vorwerk et al., J. Clin. Endocrinol. Metab. 82(7), 2368-70 (1997)) were purchased from Diagnostic Systems Laboratories (Webster, Tex.). ¹²⁵I-IGF-I and ¹²⁵I-IGF-II (2000 Ci/mmol), and the enhanced chemiluminescence (ECL) Western blotting detection reagent were purchased from Amersham Pharmacia Biotech (Amersham Pharmacia, N.J.). Fetal calf serum was obtained from Hyclone Laboratories, Inc. (Logan, Utah), whereas F12K Nutrient Mixture medium, Dulbecco's modified Eagle's Medium (DMEM) containing 4.5 g/l D-glucose, pyridoxine hydrochloride, sodium pyruvate, LipofectAMiNE PLUS and G418 (734 μg/mg) were obtained from Life Technologies (Grand Island, N.Y.). The BrdU (5-bromo-2′-deoxyuridine) Cell Proliferation ELISA (enzyme-linked immunosorbent assay), Apoptotic DNA Ladder, In Situ Cell Death Detection (TUNEL or terminal deoxynucleotidyl transferase-mediated dUTP nick end labeling assay), Cell Death Detection ELISA Plus assay kits, and the fluorescent DNA-binding dye DAPI (4′,6-diamidine-2′-phenylindole hydrochloride) were purchased from Roche Molecular Biochemicals (Indianapolis, Ind.). Recombinant human epidermal growth factor (EGF) was obtained from Sigma (St. Louis, Mo.).

Cell cultivation—CHO-K1 cells (Arai et al., J. Biol. Chem. 271(11), 6099-106 (1996)) were obtained from David Clemmons (University of North Carolina School of Medicine, Chapel Hill), whereas mink lung epithelial cells (CCL64) were obtained from Anita Roberts (National Cancer Institute) or the American Type Culture Collection (ATCC, Manassas, Va.), and PC-3 human prostate adenocarcinoma cells (Kaighn et al., Invest. Urol. 17(1), 16-23 (1979)) were obtained from the ATCC. CHO-K1 and PC-3 cells were grown in F12K medium containing 10% fetal calf serum, whereas CCL64 cells were grown in DMEM plus 10% fetal calf serum. All media contained penicillin (100 U/ml), streptomycin (100 μg/ml) and fungizone (2.5 μg/ml). Cells were grown at 37° C. in a humidified environment with 5% CO₂. Fresh cells were thawed at least every 2 months.

Construction of the expression plasmid encoding wild-type human IGFBP-3 (pRSV-Sec-BP3)—Plasmid pRSV-Sec-BP-3 expresses a fusion gene encoding the signal peptide of the immunoglobulin kappa chain and a peptide containing a 6×His/Xpress antibody recognition site/enterokinase C cleavage site upstream from the 795 nt coding region of hIGFBP-3 cDNA. First, a double-stranded oligonucleotide (5′-AGCT ATG GAG ACA GAC ACA CTC CTG CTA TGG GTA CTG CTG CTC TGG GTT CCA GGT TCC ACT GGT GAC A-3′) (SEQ ID NO: 3) encoding the IgG kappa chain signal peptide (pSecTag2, Invitrogen) with HindIII sticky ends was introduced into the HindIII site of pRc/RSV (Invitrogen) to form pRSV-Sec. The upstream HindIII site was destroyed by the single base change (T→A) at the last amino acid of the HindIII site. Next, a double-stranded DNA fragment containing the 6×His/Xpress antibody/enterokinase C sequence fused to the coding region of mature hIGFBP-3 was prepared by overlapping PCR. The 5′-fragment containing the 6×His/Xpress antibody/enterokinase sequence (CAT CAT CAT CAT CAT CAT GGT ATG GCT AGC ATG ACT GGT GGA CAG CAA ATG GGT CGG GAT CTG TAC GAC GAT GAC GAT AAG) (SEQ ID NO: 4) was amplified from pcDNA3.1/His A (Invitrogen); the 5′ end of the sense primer was extended by a HindIII sequence, and the 5′ end of the antisense primer was extended by the N-terminal 18 bp of hIGFBP-3 (GCC CCC CGA GCT CGC GCC) (SEQ ID NO: 5). The 3′ fragment contained the complete coding region of hIGFBP-3 (795 bp); the 5′ end of the sense primer was extended by the Xpress antibody/enterokinase tag, and the 5′ end of the antisense primer was extended by an XbaI sequence. Following overlapping PCR of the two fragments using the HindIII and XbaI primers, the complete HindIII-6×His-Xpress-EK-hIGFBP-3-XbaI fragment was ligated into the HindIII-XbaI gap of linearized pRSV-Sec to produce pRSV-Sec-BP3 (FIG. 1).

Construction of plasmids expressing hIGFBP-3 Mutants—Alanine substitution mutations were introduced into pRSV-Sec-BP-3 using the QuikChange Site-Directed Mutagenesis Kit (Stratagene) as described by the manufacturer. Complementary oligonucleotide primers to the same sequence containing the desired mutations were annealed to both strands of the double-stranded DNA vector (pRSV-Sec-BP3) and extended using PfuTurbo DNA polymerase to generate a mutated plasmid with staggered nicks. Following amplification, the parental DNA template was digested with DpnI endonuclease and the DpnI-treated DNA was used to transform Epicurian Coli XL 1-Blue supercompetent cells.

The double mutant R75A/L77A was formed using pRSV-Sec-BP-3 template and the oligonucleotide primers 396-cg tcg ccc gac gag gcg gca ccg gcg cag gcg ctg ctg gac gg -438 (where cga and ctg were changed to gca and gcg (bold)) (SEQ ID NO: 6). The quadruple mutant R75A/L77A/L80A/L81A was formed using an oligonucleotide containing both an R75A/L77A mutation (underlined) and an L80A/L81A mutation (where ctgctg was changed to gctgcg (bold)): 411-g gca ccg gcg cag gcg gct gcg gac ggc cgc ggg -444 (SEQ ID NO: 7). The plasmid containing six mutations (I56A/Y57A/R75A/L77A/L80A/L81A) was constructed using the R75A/L77A/L80A/L81A plasmid as template and oligonucleotides to introduce the I56A/Y57A mutations: 341-g ggc cag ccg tgc ggc gct gct acc gag cgc tgt ggc-377 (where atc tac was changed to gct gct (bold)) (SEQ ID NO: 8). The sequences of all mutations were confirmed using the DNA Sequencing Kit (PE Applied Biosystems, Foster City Calif.).

Transfection and selection of stable cell lines—CHO-K1 cells in 10-cm culture dishes were transfected with 4 μg plasmid DNA (wild-type or mutant pRSV-Sec-BP3, or empty vector pRSV-Sec), LipofectAMINE (10 μl) and PLUS reagent (15 μl) in serum-free F12K medium according to the manufacturer's instructions. After 3 h, fetal calf serum was added to a final concentration of 10% and the incubation continued for 24 h, following which G418 (1000 μg/ml) was added to select the neomycin-resistant transfected cells. After 48 h, conditioned media were examined for gene expression by immunoblotting with anti-Xpress antibody, and cells from positive transfections were replated at 1:1,000 dilution in the same selection medium. After 7 days, ˜85% of the cells had been killed. Single colonies were picked into 24-well dishes, grown to confluence in selection medium, and the medium changed to serum-free medium containing G418. After 48 h, the conditioned media were examined by immunoblotting with monoclonal antibody to the N-terminal region of hIGFBP-3. Clones with the highest expression of transfected IGFBP-3 were selected and expanded.

Collection and purification of expressed hIGFBP-3—Stably transfected CHO-K1 cells expressing wild-type or mutant hIGFBP-3 were grown to confluence in 175 cm² flasks in 20 ml of F 12K medium supplemented with 10% fetal calf serum and G418. The monolayer was washed with phosphate-buffered saline, the medium changed to serum-free F12K medium containing G418 and the cells cultured for another two days. The medium was harvested, serine protease inhibitors phenylmethylsulfonyl fluoride and 4-(2-aminoethyl)-benzenesulfonyl fluoride hydrochloride were added immediately (final concentration 0.1 mg/ml), and the medium was centrifuged to remove cell debris. The clarified medium was immediately concentrated ˜10× using Centriprep YM-10 filters (Millipore) and stored at −70° C. The cells were trypsinized and replated, and the process repeated up to 7-8 times.

Wild-type and mutant 6×His-hIGFBP-3 were purified by affinity chromatography using ProBond resin which contains immobilized nickel divalent cations. First, individual samples were immunoblotted with monoclonal antibody to the N-terminus of hIGFBP-3 to exclude samples containing 30-kDa hIGFBP-3 fragments. Then, the column (5 ml) was loaded with an equal volume of concentrated conditioned media at 0° C. It was washed with 20 mM sodium phosphate-0.5 M NaCl buffer (pH 7.8), then with pH 6.0 sodium phosphate-NaCl followed by the same buffer containing 50 mM imidazole. The column was eluted successively with pH 6.0 sodium phosphate-NaCl buffer containing 200 mM imidazole and 350 mM imidazole. The combined eluates were concentrated 10× using Centriprep YM-10 filters and desalted using a PD-10 column (Sephadex G25M; Amersham Pharmacia Biotech) equilibrated with phosphate buffered saline. Serine protease inhibitors were added again and the desalted purified samples were stored at −70° C.

Quantification of affinity-purified hIGFBP-3 samples—The concentration of hIGFBP-3 present in the affinity-purified preparations was determined by quantitative immunoblotting using N-terminal and C-terminal monoclonal antibodies to hIGFBP-3. Samples were tested at 3-4 concentrations and compared with a standard curve generated using recombinant glycosylated hIGFBP-3 (R&D Systems). The resulting autoradiographs were scanned and the signal quantified using the NIH Image program as described below. The concentration of hIGFBP-3 in the samples was determined from the linear portion of the standard curve in four assays. Results using N-terminal and C-terminal antibodies were not significantly different and were combined. Control medium was collected in parallel from CHO-K1 cells stably transfected with pRSV-Sec empty vector and subjected to the same concentration and affinity purification. The amount of empty vector control is given as equivalents of wild-type CHO-hIGFBP-3 obtained from the same volume of conditioned media in a parallel purification.

Immunoblotting—IGFBP samples were mixed with 2× Laemmli loading buffer without dithiothreitol (Bio-Rad, Hercules Calif.) and were heated at 95° C. for 5 min. The samples were separated on a 10-20% gradient SDS-PAGE (sodium dodecyl sulfate polyacrylamide gel electrophoresis) and proteins transferred onto a nitrocellulose membrane. After blocking with 1× phosphate-buffered saline-10% non-fat dry milk (4° C., overnight), the membrane was incubated with a 1:10,000 dilution of monoclonal antibody to the N-terminus or C-terminus of hIGFBP-3 for 2 h. The membrane was washed three times with phosphate-buffered saline plus 0.1% Tween 20, incubated with anti-mouse IgG-horseradish peroxidase (1:5000 dilution; Santa Cruz Biotechnology), and processed for detection using an enhanced chemiluminescence detection system. The membrane was sealed in a plastic bag and exposed to high sensitivity X-ray film. The resulting autoradiograph was scanned using an ArcusII scanner and Foto Look 2.07.02 software. Signal intensities were analyzed using the NIH Image program.

Ligand blotting—Following immunoblotting, the membranes were washed with 10 mM Tris-Cl (pH 7.4)-0.15 M NaCl-3% Nonidet-P40-0.5 mg/ml sodium azide (22° C., 1 h), and then incubated in 5 ml of the same buffer containing 400,000 cpm ¹²⁵I-IGF-I or ¹²⁵ I-IGF-II (3 h, room temperature) (Hossenlopp et al., Anal. Biochem. 154(1), 138-43 (1986); Yang et al., Handbook of Endocrine Research Techniques, pp. 181-204, Academic Press, San Diego (1993)). After washing three times (15 min each) with the same buffer without radioligand, the membrane was exposed to high sensitivity film at −70° C. Ligand blotting confirmed that the 24-kDa IGFBP4 that was present in the media of nontransfected CHO-K1 cells had been removed by affinity chromatography.

Binding of ¹²⁵I-IGF-I and ¹²⁵I-IGF-II in solution —¹²⁵I-IGF-I or ¹²⁵I-IGF-II (˜25,000 cpm) was incubated overnight at 4° C. with different concentrations of recombinant hIGFBP-3 standard (R&D Systems), or purified wild-type or mutant CHO-hIGFBP-3 in 0.4 ml phosphate-buffered saline supplemented with 0.2% fatty acid-free bovine serum albumin. Following the addition of 0.5 ml of a 5% suspension of activated charcoal (Sigma) to adsorb unbound IGF tracer, the samples were centrifuged. Radioactivity bound to hIGFBP-3 remained in the charcoal supernate and was quantified in a gamma counter (Yang et al. (1993), supra). DNA synthesis—DNA synthesis was measured in CCL64 cells by the incorporation of the thymidine analog BrdU into newly synthesized as previously described (Wu et al. (2000), supra). Quiescent cells in serum-free medium were stimulated to synthesize DNA by adding EGF. EGF was used to stimulate proliferation instead of serum to avoid introducing IGFs. In brief, the cells were plated in 96-well microtiter plates (30,000 cells/well) in 0.2 ml of DMEM containing 10% fetal calf serum, and incubated for 3 h at 37° C. The medium was replaced with serum-free DMEM supplemented with 0.5% bovine serum albumin (Sigma, radioimmunoassay grade), and the incubation continued for another 3 h. EGF (20 ng/ml) and the indicated concentrations of hIGFBP-3 were added, and the incubation continued overnight. BrdU (10 μM) was added for 2-3 h, the cells were fixed, and BrdU incorporation was quantified by an immunocolorimetric assay using monoclonal antibody to BrdU conjugated to peroxidase. Triplicate points were examined. The absorbance at 450 nm was measured in a scanning multiwell spectrophotometer.

Trypan Blue staining of nonviable cells—PC-3 cells were plated in 12-well culture dishes (50-130,000 cells/well) and grown to confluence (24 h) in F12K medium supplemented with 10% fetal calf serum. The medium was changed to serum-free medium for 24 h, and then replaced with fresh serum-free medium containing wild-type CHO-hIGFBP-3 (1 μg/ml), 6m-hIGFBP-3 (1 μg/ml) or protein purified from pRSV-Sec empty vector transfectants (equivalent to 2 μg/ml of wild-type CHO-hIGFBP-3); Leu⁶⁰-IGF-I was added where indicated. After 24, 48 or 72 h incubation, floating cells in the medium were sedimented and resuspended; adherent cells were dissociated with trypsin and resuspended. Trypan blue (0.4%) was added to the suspensions of floating and attached cells and incubated for 10 min. The total number of cells and the number of non-viable cells stained with trypan blue were counted in a hemocytometer.

DNA ladder—PC-3 cells were plated in a 10-cm culture dish (600,000 cells/well) in serum-supplemented F12K medium and grown to confluence (24 h). The medium was changed to serum-free medium for 24 h and was replaced with fresh serum-free medium containing wild-type CHO-hIGFBP-3 (1 μg/ml), 6m-hIGFBP-3 (1 μg/ml) or protein from pRSV-Sec empty vector transfectants (equivalent to 2 μg/ml of wild-type CHO-hIGFBP-3). After 72 h incubation, the cells were lysed, and the DNA purified and analyzed on 1% agarose gels containing ethidium bromide using the Apoptotic DNA Ladder Kit according to the manufacturer's instructions. In brief, the cells were lysed with 3 M guanidine hydrochloride-5 mM urea-10% Triton X-100, extracted with isopropanol, and the extract was applied to filter tubes containing a glass fiber fleece and centrifuged. After washing, the nucleic acids bound to the glass fibers were eluted with 10 mM Tris-HCl, pH 8.5, prewarmed to 70° C., and analyzed by agarose gel electrophoresis and UV photography. A ladder pattern of multiples of 180 bp nucleosomal subunits is generated in apoptotic cells.

DAPI-staining of nuclear DNA—PC-3 cells were plated in a 6-well culture dish (200,000 cells/dish) and grown to 60% confluence in F12K medium containing 10% fetal calf serum. The medium was changed to serum-free medium for 24 h and was replaced with fresh serum-free medium containing wild-type CHO-hIGFBP-3 (1 μg/ml), 6m-hIGFBP-3 (1 μg/ml) or protein from pRSV-Sec empty vector transfectants (equivalent to 2 μg/ml of wild-type CHO-hIGFBP-3) for 72 h. The cells were washed once with 1 μg/ml DAPI-methanol, incubated with DAPI-methanol (15 min, 37° C.), washed with methanol and examined by fluorescence microscopy. DAPI is a fluorescent dye that binds selectively to DNA. Nuclear condensation and fragmentation is characteristic of apoptotic cells.

TUNEL assay—Cleavage of genomic DNA into oligonucleosomes during apoptosis was identified in individual cells by labeling 3′OH termini using terminal deoxynucleotidyl transferase and fluorescein-labeled dUTP substrate (TUNEL assay).

PC-3 cells (30,000 cells) were plated on 8-well chamber slides in serum-supplemented F12K medium and grown to 80% confluence (24 h). The medium was changed to serum-free medium, and after 24 h was replaced with fresh serum-free medium containing wild-type CHO-hIGFBP-3 (1 μg/ml), 6m-hIGFBP-3 (1 μg/ml), or purified medium from cells transfected with pRSV-Sec empty vector (equivalent to 2 μg/ml of wild-type CHO-hIGFBP-3). After 72 h incubation, the cells were fixed with 2% paraformaldehyde, washed 3 times with phosphate-buffered saline, permeabilized with 0.1% Triton X-100 on ice, and incubated with the TUNEL reaction mixture in a humidified chamber (1 h, 37° C.). Cells incorporating labeled dUTP were identified by fluorescence microscopy and photographed.

ELISA assay of histone-associated DNA fragments—This assay measures histone-bound DNA fragments generated by internucleosomal cleavage in the cytosol of apoptotic cells. PC-3 cells (10,000 cells/well) were grown to 80% confluence in 96 well culture plates in serum-supplemented F12K medium. After 24 h incubation in serum-free medium, the indicated hIGFBP-3 preparations were added at different concentrations for 72 h. The cell membranes were lysed according to the manufacturer's instructions and the supernates added to streptavidin-coated microplates. Biotin-labeled anti-histone (to bind the histone component of the nucleosomes and fix the complex to the plate) and anti-DNA peroxidase (to bind to nucleosomal DNA) were added and the incubation continued for 2 h. After washing, the amount of nucleosome DNA was determined photometrically after addition of 2,2′-azino-di(3-ethyl-benzthiazoline-sulfonate) peroxidase substrate for 30 min. Absorbance was determined at 405 nm and 490 nm (substrate blank).

### Example 1

This example describes the construction and characterization of mutants of hIGFBP-3 that do not bind IGF-I and IGF-II.

Candidate mutations that might decrease the binding of IGF-I and IGF-II to hIGFBP-3 were designed. Alanine was substituted for the native amino acids at 6 positions in hIGFBP-3, i.e., Ile56, Tyr57, Arg75, Leu77, Leu80, and Leu81.

Wild-type hIGFBP-3 and mutant hIGFBP-3 containing the six alanine substitutions (I56A, Y57A, R75A, L77A, L80A, and L81A; 6m-hIGFBP-3) were expressed in CHO-K1 cells as secreted proteins containing an N-terminal polyhistidine tag to allow purification by nickel cation affinity chromatography. Different amounts of the purified proteins and recombinant hIGFBP-3 standard were fractionated using SDS-PAGE and examined by immunoblotting with monoclonal antibodies to the N- and C-terminal domains of hIGFBP-3 and by ligand blotting with ¹²⁵I-IGF-I or ¹²⁵I-IGF-II. The three proteins were recognized by monoclonal antibodies to the N-terminal and C-terminal epitopes.

When the same immunoblots were incubated with ¹²⁵I-IGF-I or ¹²⁵I-IGF-II, dose-dependent binding was observed to the recombinant hIGFBP-3 standard and to wild-type CHO-hIGFBP-3; by contrast, neither radioligand bound to similar concentrations of 6m-hIGFBP-3. Similarly, CHO-hIGFBP-3 mutated at only four (R75A, L77A, L80A, and L81A; 4m-hIGFBP-3) or two (R75A and L77A; 2m-hIGFBP-3) of the six sites did not bind ¹²⁵I-IGF-I or ¹²⁵I-IGF-II on ligand blot

The 6m-, 4m- and 2m-hIGFBP-3 mutant proteins also were unable to bind ¹²⁵I-IGF-I or ¹²⁵I-IGF-II in a solution binding assay which did not expose them to denaturing conditions. Dose-dependent binding of ¹²⁵I-IGF-I or ¹²⁵I-IGF-II was observed with recombinant hIGFBP-3 standard or wild-type CHO-hIGFBP-3, reaching a maximum of 70-80% of input radioactivity bound. By contrast, only negligible binding was observed with any of the three mutants at concentrations as high as 200 ng/ml, less than the binding observed to 80-fold lower concentrations of wild-type CHO-hIGFBP-3. Thus, the mutant hIGFBP-3 molecules have profoundly decreased ability to bind IGF-I and IGF-II.

### Example 2

This example demonstrates that mutants of human IGFBP-3 do not bind IGF-I and IGF-II, yet still inhibit DNA synthesis in mink lung epithelial cells.

Non-glycosylated recombinant hIGFBP-3 expressed in E.coli inhibited DNA synthesis in CCL64 mink lung epithelial cells in serum-free medium (Wu et al. (2000), supra). The inhibition was considered to be IGF-independent, since CCL64 cells do not synthesize functionally significant levels of IGF-I or IGF-II, and IGF-I does not stimulate CCL64 DNA synthesis. Dose-dependent inhibition of DNA synthesis was observed not only with glycosylated recombinant hIGFBP-3 reference standard and wild-type CHO-hIGFBP-3, but also with the nonbinding hIGFBP-3 mutant proteins containing 2, 4 or 6 mutations. No inhibition was observed with equivalent amounts of conditioned media purified from nontransfected CHO-K1 cells. Thus, the hIGFBP-3 mutants retain the ability to inhibit DNA synthesis in mink lung epithelial cells even though they do not bind IGFs. These results provide strong independent confirmation of our previous conclusion that inhibition of CCL64 DNA synthesis by wild-type hIGFBP-3 is IGF-independent (Wu et al. (2000), supra).

Free hIGFBP-3 inhibits CCL64 DNA synthesis but hIGFBP-3 complexed to IGF-I does not (Wu et al. (2000), supra), presumably because IGF-I induces a conformational change in IGFBP-3 when it binds to it. Since 6m-hIGFBP-3 cannot bind IGF-I, coincubation with IGF-I should not affect its ability to inhibit CCL64 cell DNA synthesis. As in the previous study, Leu⁶⁰-IGF-I, an IGF-I analogue in which leucine is substituted for tyrosine at position 60 (Bayne et al., J. Biol. Chem. 265(26), 15648-52 (1990)), was used instead of native IGF-I, since the analogue binds to hIGFBP-3 but has low affinity for and does not activate the IGF-I receptor. As expected, coincubation with Leu⁶⁰-IGF-I (at 0.5 or 2 μg/ml) abolished the inhibition of DNA synthesis caused by 2 μg/ml wild-type CHO-hIGFBP-3 but did not decrease the inhibition induced by 6m-hIGFBP-3. These results demonstrate directly that Leu⁶⁰-IGF-I must bind to hIGFBP-3 to decrease its ability to inhibit CCL64 DNA synthesis.

### Example 3

This example demonstrates that a mutant of human IGFBP-3 induces apoptosis in PC-3 human prostate cancer cells.

The ability of wild-type CHO-hIGFBP-3, 6m-hIGFBP-3, or media from CHO-K1 cells transfected with empty vector to kill serum-deprived PC-3 cells was examined. After 72 h, approximately 50% of the cells recovered after incubation with wild-type or 6m-CHO-hIGFBP-3 had detached from the monolayer, whereas <0.1% of the cells recovered after incubation with media from empty vector transfectants were floating. Over 86% of the floating cells from the wild-type or 6m-CHO-hIGFBP-3 incubations were nonviable (i.e., stained with trypan blue), whereas <20% of cells that remained attached to the culture dish were dead, whether or not they had been incubated with hIGFBP-3. Thus, incubating serum-deprived PC-3 cells with either wild-type CHO-hIGFBP-3 or 6m-hIGFBP-3 promoted the detachment of cells from the monolayer and greatly increased the percentage of nonviable cells.

As with the inhibition of CCL64 cell DNA synthesis by hIGFBP-3, only free hIGFBP-3 induced PC-3 cell death. Coincubation with Leu⁶⁰-IGF-I markedly decreased the percentage of floating PC-3 cells treated with hIGFBP-3 standard or wild-type CHO-hIGFBP-3 that were dead from ˜82% to ˜14%. By contrast, coincubation with Leu⁶⁰-IGF-I did not decrease the percentage of floating PC-3 cells treated with 6m-hIGFBP-3 that were dead (86% without Leu⁶⁰-IGF-1, 80% with Leu⁶⁰-IGF-1). Thus, Leu⁶⁰-IGF-I must bind to hIGFBP-3 to prevent it from inducing PC-3 cell death.

The increased death of PC-3 cells incubated with wild-type or 6m-CHO-hIGFBP-3 reflects increased apoptosis. This was demonstrated using several indices of apoptosis-induced DNA fragmentation. Agarose gel electrophoresis of DNA preparations from cells incubated with wild-type or 6m-CHO-hIGFBP-3, but not from control cells, revealed a ladder of DNA fragments of different sizes that represent oligonucleosomes containing different numbers of nucleosomes. Nuclear staining of individual cells with the fluorescent dye DAPI revealed DNA fragmentation and condensation characteristic of apoptosis in cells treated with wild-type CHO-hIGFBP-3 or 6m-hIGFBP-3. Apoptosis also was seen in individual cells using the TUNEL assay in which terminal deoxynucleotidyl transferase catalyzes the addition of fluorescein-dUTP to the free 3′-OH ends of DNA fragments generated by apoptosis. Numerous cells incorporating the fluorescent nucleotide were evident by fluorescent microscopy of cells treated with wild-type CHO-hIGFBP-3 or 6m-hIGFBP-3 but not with media from empty vector transfectants. Finally, using a quantitative ELISA assay, the abundance of cytosolic histone-bound DNA fragments was increased approximately 10-fold in cells incubated with 1 μg/ml wild-type CHO-hIGFBP-3 or 6m-hIGFBP-3 compared with media from empty vector transfectants. Stimulation was observed at 30 ng/ml, and the dose response curves with the native and mutant proteins were superimposable. Thus, the stimulation of PC-3 cell apoptosis by 6m-hIGFBP-3 and wild-type CHO-hIGFBP-3 is similar in magnitude and concentration dependence, suggesting that IGF-independent mechanisms are major contributors to the induction of apoptosis in PC-3 cells by IGFBP-3.

All of the references cited herein, including patents, patent applications, and publications, are hereby incorporated in their entireties by reference.

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the claims.

What is claimed is:

1. An isolated or purified nucleic acid molecule encoding a human insulin-like growth factor binding protein-3 (IGFBP-3) comprising a mutation at amino acid Y57 and optionally a mutation of one or more amino acids of the insulin-like growth factor (IGF)-binding domain of IGFBP-3, wherein the IGFBP-3 can inhibit DNA synthesis, can induce apoptosis, and binds to neither human insulin-like growth factor-I (IGF-I) nor human insulin-like growth factor-II (IGF-II).

2. The isolated or purified nucleic acid molecule of claim 1, wherein the one or more amino acids are selected from the group consisting of I56, R75, L77, L80, and L81.

3. The isolated or purified nucleic acid molecule of claim 2, wherein the mutation is a substitution of at least one of the amino acids selected from the group consisting of I56, Y57, R75, L77, L80, and L81 with another amino acid that compromises the ability of IGFBP-3 to bind to IGF-I and IGF-II.

4. The isolated or purified nucleic acid molecule of claim 3, wherein the amino acid that compromises the ability of IGFBP-3 to bind to IGF-I and IGF-II is alanine.

5. The isolated or purified nucleic acid molecule of claim 4, wherein all of I56, Y57, R75, L77, L80, and L81 are substituted with alanine.

6. A vector comprising the isolated or purified nucleic acid molecule of claim 1.

7. A vector comprising the isolated or purified nucleic acid molecule of claim 2.

8. A vector comprising the isolated or purified nucleic acid molecule of claim 3.

9. A vector comprising the isolated or purified nucleic acid molecule of claim 4.

10. A vector comprising the isolated or purified nucleic acid molecule of claim 5.

11. A cell comprising and expressing the isolated or purified nucleic acid molecule of claim 1, optionally in the form of a vector.

12. A cell comprising and expressing the isolated or purified nucleic acid molecule of claim 2, optionally in the form of a vector.

13. A cell comprising and expressing the isolated or purified nucleic acid molecule of claim 3, optionally in the form of a vector.

14. A cell comprising and expressing the isolated or purified nucleic acid molecule of claim 4, optionally in the form of a vector.

15. A cell comprising and expressing the isolated or purified nucleic acid molecule of claim 5, optionally in the form of a vector.

16. An isolated or purified human IGFBP-3 polypeptide molecule comprising a mutation at amino acid Y57 and optionally a mutation of one or more amino acids of the IGF-binding domain of IGFBP-3, wherein the IGFBP-3 polypeptide molecule can inhibit DNA synthesis, can induce apoptosis, and binds to neither human insulin-like growth factor-I (IGF-I) nor human insulin-like growth factor-II (IGF-II).

17. The isolated or purified human IGFBP-3 polypeptide molecule of claim 16, wherein the one or more amino acids are selected from the group consisting of I56, R75, L77, L80, and L81.

18. The isolated or purified human IGFBP-3 polypeptide molecule of claim 17, wherein the human IGFBP-3 polypeptide molecule comprises a substitution of at least one of the amino acids selected from the group consisting of I56, Y57, R75, L77, L80, and L81 with another amino acid that compromises the ability of IGFBP-3 to bind to IGF-I and IGF-II.

19. The isolated or purified human IGFBP-3 polypeptide molecule of claim 18, wherein the amino acid that compromises the ability of IGFBP-3 to bind to IGF-I and IGF-II is alanine.

20. The isolated or purified human IGFBP-3 polypeptide molecule of claim 19, wherein all of I56, Y57, R75, L77, L80, and L81 are substituted with alanine.

21. A composition comprising the isolated or purified human IGFBP-3 polypeptide molecule of claim 16.

22. A composition comprising the isolated or purified human IGFBP-3 polypeptide molecule of claim 17.

23. A composition comprising the isolated or purified human IGFBP-3 polypeptide molecule of claim 18.

24. A composition comprising the isolated or purified human IGFBP-3 polypeptide molecule of claim 19.

25. A composition comprising the isolated or purified human IGFBP-3 polypeptide molecule of claim 20.

26. A method of inducing apoptosis in a cell, which method comprises administering to the cell:
(a) an isolated or purified nucleic acid molecule encoding a human IGFBP-3 comprising a mutation at amino acid Y57 and optionally a mutation of one or more amino acids of the IGF-binding domain of IGFBP-3, wherein the IGFBP-3 can inhibit DNA synthesis, can induce apoptosis, and binds to neither human insulin-like growth factor-I (IGF-I) nor human insulin-like growth factor-II (IGF-II), or
(b) an isolated or purified human IGFBP-3 polypeptide molecule comprising a mutation at amino acid Y57 and optionally a mutation of one or more amino acids of the IGF-binding domain of IGFBP-3, wherein the IGFBP-3 polypeptide molecule can inhibit DNA synthesis, can induce apoptosis, and binds to neither human insulin-like growth factor-I (IGF-I) nor human insulin-like growth factor-II (IGF-II),
in an amount sufficient to induce apoptosis in the cell, whereupon apoptosis is induced in the cell.

27. The method of claim 26, wherein the cell is in a host.

28. The method of claim 27, wherein the host is a mammal.

29. The method of claim 28, wherein the mammal is a human.

30. The method of claim 27, wherein the host is afflicted with a cancer, whereupon the cancer is effectively treated in the host.

31. The method of claim 30, wherein the cancer is a cancer selected from the group consisting of prostate cancer, colorectal cancer, lung cancer, and childhood-onset leukemia.


SEQUENCE LISTING (8 sequences)

SEQ ID NO: 1 - protein, 34 aa, Homo sapiens
EGQACGVYTERCAQGLRCLPRQDEEKPLHALLHG

SEQ ID NO: 2 - protein, 34 aa, Homo sapiens
EGQPCGIYTERCGSGLRCQPSPDEARPLQALLDG

SEQ ID NO: 3 - DNA, 68 bp, Homo sapiens
agctatggagacagacacactcctgctatgggtactgctgctctgggttccaggttccactggtgaca

SEQ ID NO: 4 - DNA, 81 bp, Homo sapiens
catcatcatcatcatcatggtatggctagcatgactggtggacagcaaatgggtcgggatctgtacgacgatgacgataag

SEQ ID NO: 5 - DNA, 18 bp, Homo sapiens
gccccccgagctcgcgcc

SEQ ID NO: 6 - DNA, 43 bp, Homo sapiens
cgtcgcccgacgaggcggcaccggcgcaggcgctgctggacgg

SEQ ID NO: 7 - DNA, 34 bp, Homo sapiens
ggcaccggcgcaggcggctgcggacggccgcggg

SEQ ID NO: 8 - DNA, 37 bp, Homo sapiens
gggccagccgtgcggcgctgctaccgagcgctgtggc